(19) United States
(12) Patent Application Publication
(10) Pub. No.: US 2025/0027896 A1
(43) Pub. Date: Jan. 23, 2025

(54) ZINC OXIDE SYNTHESIS TECHNOLOGY THAT SIMULTANEOUSLY PRODUCES UNIFORM GOLD NANOPARTICLE FORMATION AND SURFACE DEFECTS

(71) Applicant: Agency for Defense Development, Yuseong-gu (KR)

(72) Inventors: MinKyung LEE, Yuseong-gu (KR); MyungKyu PARK, Yuseong-gu (KR)

(21) Appl. No.: 18/760,854

(22) Filed: Jul. 1, 2024

(30) Foreign Application Priority Data
Jul. 19, 2023 (KR) 10-2023-0093658

(51) Int. Cl. G01N 27/12; C01G 9/02; G01N 33/00

(52) U.S. Cl. CPC G01N 27/127; C01G 9/02; G01N 33/0027; C01P 2002/72; C01P 2002/82; C01P 2002/85; C01P 2004/04; C01P 2006/60

(57) ABSTRACT

Provided is a porous metal-zinc oxide nanosheet comprising metal nanoparticles formed on the surface.

### CROSS-REFERENCE TO RELATED APPLICATION

This application claims the benefit of Korean Patent Application No. 10-2023-0093658, filed on Jul. 19, 2023, in the Korean Intellectual Property Office, the entire disclosure of which is incorporated herein by reference for all purposes.

### BACKGROUND

### 1. Field of the Invention

The present disclosure relates to a zinc oxide synthesis technology that simultaneously produces uniform gold nanoparticle formation and surface defects.

### 2. Description of the Related Art

For decades, a metal oxide semiconductor (MOS) having wide electronic band gap energy has been used as an electrochemical sensing material for analyzing gaseous compounds. In particular, when vapor molecules are adsorbed on the surface of a metal oxide, electron exchange occurs through a series of chemical reactions. Compared to conventional analysis methods, a gas sensor based on the metal oxide semiconductor has advantages of miniaturization, low manufacturing cost, and low power consumption due to the simplicity of a measurement system. Recently, the successful detection of DMMP and 2-CEES, which are nerve agent (GB) and blister agent (HD)-like agents, has been often performed using chemically modified metal oxide semiconductors obtained through doping, morphological control, heteroatom junction formation, and catalytic domain induction. Although these approaches have excellent sensing performance for target gases, the generation of undesired signals from interfering compounds remains a major challenge.

Heterostructure formation between noble metals and oxide sensing materials is an essential approach for improving selectivity because a specific molecular structure has a high affinity with metal surfaces.

For example, gold is known to have an inert surface in a bulk scale, but may be modified to a nanoscale through ligand chemistry based on a thiol (R—S—H) functional group. R—S—H is known to have a strong affinity with the Au (111) surface and be converted to a chemically trapped thiyl radical (R—S·). The gold-sulfur interaction is used for wide applications, such as biosensing, molecular electronics, drug delivery, and nanopatterning.

The aforementioned background art is included or obtained by the present inventors in the process of deriving the disclosure of the present disclosure, and may not necessarily be known art disclosed to the general public prior to the present application.

### SUMMARY

Embodiments provide a zinc oxide synthesis technology that simultaneously produces uniform gold nanoparticle formation and surface defects.

Specifically, embodiments provide a method for distributing gold nanoparticles at high density on a metal oxide surface within 1 minute by a simple microwave radiation approach and supplying narrow particle size distribution and strong structural binding.

However, technical goals to be achieved are not limited to those described above, and other goals not mentioned above are clearly understood by one of ordinary skill in the art from the following description.

According to an aspect, there is provided a porous metal-zinc oxide nanosheet including metal nanoparticles formed on the surface.

According to one example, the diameter of the metal nanoparticles may be 0.1 nm to 5 nm, and a ratio of the metal nanoparticles on the surface of the porous metal-zinc oxide nanosheet may be 1.0 wt % or more.

According to one example, the porous metal-zinc oxide nanosheet may include oxygen defects on the surface, and a molar ratio of the oxygen defects may be 5% to 30%.

According to one example, the porous metal-zinc oxide nanosheet may further include an electron depletion layer.

According to one example, a thickness ratio of the porous metal-zinc oxide nanosheet and the electron depletion layer may be 1:1 to 8:1.

According to one example, the metal nanoparticles may be formed in an area with a depth corresponding to 0% to 10% from the surface of a distance from the surface to the center of the porous metal-zinc oxide nanosheet.

According to one example, the metal nanoparticles on the surface of the porous metal-zinc oxide nanosheet may be disposed at intervals of 0 nm to 10 nm.

According to one example, the metal nanoparticles may include at least one selected from the group consisting of Pt, Pd, Rh, Ru, Ni, Co, Cr, Ir, Au, Ag, Pb, Fe, Cu, Al, Ti, Ce, and Cd.

According to one example, pores of the porous metal-zinc oxide nanosheet may be included in 5 vol % to 30 vol % of the total volume of the porous metal-zinc oxide nanosheet.

According to another aspect, there is provided a method for manufacturing a porous metal-zinc oxide nanosheet including: preparing a zinc oxide nanosheet; forming a porous zinc oxide nanosheet by heat-treating the zinc oxide nanosheet; forming a mixture by mixing the porous zinc oxide nanosheet and a metal precursor; and forming metal nanoparticles on the surface of the porous zinc oxide nanosheet by irradiating the mixture with microwaves.

According to one example, in the irradiating of the mixture with microwaves, the microwaves may be irradiated at 750 W to 1000 W output for 10 sec to 60 sec.

According to another aspect, there is provided a gas sensor including a chip; and the porous metal-zinc oxide nanosheet formed on the chip.

According to one example, the gas sensor may sense at least one selected from the group consisting of a blister agent (HD), 2-CEES, H₂S, CH₄, CO₂, CO, SO₂, NO₂, Nh₃, Benzene, Toluene, Xylene, DMMP and 2-CEEC.

According to one example, the gas sensor may sense gas at a limit concentration of 0.1 ppm at 300° C. to 500° C., and may include a recovery time of 5 sec to 60 sec.

According to one example, the gas sensor may have responsitivity of 50 or more at 20% humidity, and responsitivity of 30 or more at 80% humidity.

According to embodiments, it is possible to provide a zinc oxide synthesis technology that simultaneously produces uniform gold nanoparticle formation and surface defects.

According to embodiments, the zinc oxide synthesis technology that simultaneously produces uniform gold nanoparticle formation and surface defects may clearly demonstrate the usefulness of a simple and fast approach in which gold nanoparticles are uniformly immobilized on the oxide surface in a short time of less than 1 minute in a metal oxide nanosheet, thereby greatly improving gas sensing properties.

In addition, the present disclosure may propose an efficient method for developing heteroatomic materials for various electrochemical applications in energy and catalysts by binding a uniform noble metal with a metal semiconductor oxide through microwave synthesis on the surface of the metal oxide.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic diagram illustrating a gas sensing mechanism according to Example and Comparative Example of the present disclosure; FIG. 1A a state without gold nanoparticles, FIG. 1B a state in which large gold nanoparticles are formed at a rare density, FIG. 1C a state in which nano-sized gold nanoparticles are densely immobilized, FIG. 1D a schematic diagram illustrating a gas sensing mechanism on the surface of a porous zinc oxide nanosheet, FIG. 1E a schematic diagram illustrating a gas sensing mechanism on the surface of a porous gold-zinc oxide nanosheet, FIG. 1F changes in potential barrier of a porous zinc oxide nanosheet, and FIG. 1G changes in potential barrier of a porous gold-zinc oxide nanosheet;

FIG. 2 illustrates FIG. 2A a schematic diagram of a manufacturing process of a porous gold-zinc oxide nanosheet containing gold nanoparticles formed on the surface, FIG. 2B a surface element analysis result of a porous gold-zinc oxide nanosheet using energy-dispersive X-ray spectroscopy, FIG. 2C a high-resolution transmission electron microscopy image of a porous zinc oxide nanosheet, FIG. 2D a high-resolution transmission electron microscopy image of a porous gold-zinc oxide nanosheet, and FIG. 2E a size distribution of gold nanoparticles formed on the surface of a porous gold-zinc oxide nanosheet, according to Example and Comparative Example of the present disclosure;

FIG. 3 illustrates FIG. 3A crystallographic analysis results of a porous gold-zinc oxide nanosheet and a porous zinc oxide nanosheet using X-ray diffraction, FIG. 3B a result of chemical bond state analysis of a porous zinc oxide nanosheet and a porous gold-zinc oxide nanosheet obtained from movement of zinc oxide lattice position after microwave irradiation, peak expansion after immobilization of gold nanoparticles, and high-resolution X-ray photoelectron spectroscopy, FIG. 3C zinc binding energy before and after gold nanoparticle immobilization, FIG. 3D oxygen binding states of a porous zinc oxide nanosheet and a porous gold-zinc oxide nanosheet, and FIG. 3E a binding state of gold nanoparticles immobilized on the surface of the porous gold-zinc oxide nanosheet, according to Example and Comparative Example of the present disclosure;

FIG. 4 illustrates FIG. 4A a result of measuring photoluminescence (PL) of a porous zinc oxide nanosheet and a porous gold-zinc oxide nanosheet and FIG. 4B a result of Raman shift observation after immobilizing gold nanoparticles, according to Example and Comparative Example of the present disclosure;

FIG. 5 illustrates results of analyzing bandgap structures of a porous zinc oxide nanosheet and a porous gold-zinc oxide nanosheet according to Example and Comparative Example of the present disclosure; FIG. 5A kinetic energy of emitted electrons, FIG. 5B binding energy of electrons measured by ultraviolet photoelectron spectroscopy, FIG. 5C a band gap energy measurement result calculated from Tauc-plot obtained by UV-vis-NIR spectroscopy, and FIG. 5D expected band structures of a porous zinc oxide nanosheet and a porous gold-zinc oxide nanosheet;

FIG. 6 illustrates FIG. 6A a temperature-dependent resistance change curve upon exposure to 10 ppm 2-CEES as a result of evaluating gas sensing performance of a porous zinc oxide nanosheet and a porous gold-zinc oxide nanosheet, FIG. 6B a result of summarizing gas responsivity of a porous zinc oxide nanosheet and a porous gold-zinc oxide nanosheet produced by a hydrothermal synthesis method and a porous gold-zinc oxide nanosheet irradiated with microwaves, FIG. 6C a resistance change curve of a gas sensor including a porous gold-zinc oxide nanosheet exposed to various concentrations of 2-CEES gas by gas concentration-dependent gas sensing performance of the gas sensor including the porous gold-zinc oxide nanosheet, FIG. 6D gas responsivity according to a gas concentration obtained through FIG. 6C, FIG. 6E a result of evaluating sensing performance reproducibility by repeating gas exposure 20 times for 12 hours, FIG. 6F a result of measuring gas sensing performance of gas sensors including a porous gold-zinc oxide nanosheet and a porous zinc oxide nanosheet under various humidity conditions, and FIG. 6G sensing selectivity of gas sensors including porous gold-zinc oxide nanosheets for various gaseous compounds, according to Example and Comparative Example of the present disclosure; and

FIG. 7 illustrates FIG. 7A a photograph of a portable sensor module with dimension 34 mm×22 mm and weight 4.82 g, FIG. 7B a brief diagram of a gas sensing experiment, and FIG. 7C a resistance change curve of gas sensors including porous gold-zinc oxide nanosheets in various concentrations of 2-CEES and DMMP, as evaluation of sensing performance of a porous gold-zinc oxide nanosheet mounted on a portable sensor board according to Example and Comparative Example of the present disclosure.

### DETAILED DESCRIPTION

Hereinafter, examples will be described in detail with reference to the accompanying drawings. However, since various modifications may be made to examples, the scope of the present disclosure is not limited or restricted by these examples. It should be understood that all modifications, equivalents and substitutes for examples are included in the scope of the present disclosure.

The terms used in examples are used for the purpose of description only, and should not be construed to be limited. The singular expression includes the plural expression unless the context clearly dictates otherwise. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which examples pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the examples, a detailed description of related known technologies will be omitted if it is determined that they unnecessarily make the gist of the examples unclear. In describing the components of the examples of the present disclosure, terms including first, second, A, B, (a), (b), and the like may be used. These terms are just intended to distinguish the components from other components, and the terms do not limit the nature, sequence, or order of the components.

Components included in any one example and components having a common function will be described using the same names in other examples. Unless otherwise stated, descriptions described in any one example may also be applied to other examples, and detailed descriptions in the overlapping range will be omitted.

Hereinafter, a zinc oxide synthesis technology that simultaneously produces uniform gold nanoparticle formation and surface defects of the present disclosure will be described in detail with reference to examples and drawings. However, the present disclosure is not limited to these examples and drawings.

A porous metal-zinc oxide nanosheet of the present disclosure includes metal nanoparticles formed on the surface.

According to one example, the metal nanoparticles may be formed on the surface of the porous metal-zinc oxide nanosheet by irradiating microwaves.

According to one example, the microwave irradiation approach may provide high density decoration, narrow particle size distribution, and strong structural immobilization of metal nanoparticles on the surface of the porous metal-zinc oxide nanosheet within 1 minute.

According to one example, the immobilized metal nanoparticles may affect the lattice structure of the porous metal-zinc oxide nanosheet by inducing many defect sites and oxygen defects. A thicker electron depletion layer is formed due to a large number of oxygen defects, which may contribute to a very large resistance change observed upon exposure to a target molecule.

For example, the gold nanoparticles immobilized on the surface of the porous metal-zinc oxide nanosheet may provide sulfur-selective detection characteristics by using a high affinity between Au (111) and thiyl radicals. In addition, effective adsorption of a decomposed 2-CEES product on the gold nanoparticles may promote a Mars-van Krevelen reaction mechanism for complete oxidation.

In the present disclosure, it may be expected that an anchoring effect of noble metals on the surface of metal oxide through microwaves may provide an excellent aspect for the development of heteroatomic materials for various electrochemical applications in energy, catalyst, and sensor fields.

According to one example, the diameter of the metal nanoparticles may be 0.1 nm to 5 nm, and a ratio of the metal nanoparticles on the surface of the porous metal-zinc oxide nanosheet may be 1.0 wt % or more.

According to one example, the diameter of the metal nanoparticles may be 0.1 nm to 5 nm; 0.1 nm to 4 nm; 0.1 nm to 3 nm; 0.1 nm to 2 nm; 0.1 nm to 1 nm; 0.5 nm to 5 nm; 0.5 nm to 4 nm; 0.5 nm to 3 nm; 0.5 nm to 2 nm; 0.5 nm to 1 nm; 1 nm to 5 nm; 1 nm to 4 nm; 1 nm to 3 nm; and 1 nm to 2 nm.

According to one example, when the diameter of the metal nanoparticles is less than 0.1 nm, in the case of a gas sensor containing a metal oxide operating at a high temperature for a long time, grain growth occurs and the resistance value and sensitivity of the gas sensor are changed to deteriorate reproducibility, and when the size of the metal nanoparticles is smaller than that of gas molecules, responsivity may be reduced due to the absence of reaction sites of the gas molecules. When the diameter of metal nanoparticles is greater than 5 nm, aggregation of the metal nanoparticles becomes very large, and when the metal nanoparticles cover the entire surface, a change in the electron depletion layer is relatively small, which may reduce gas responsivity, and when the metal nanoparticles do not cover the surface uniformly and densely and exceed a certain amount, the surface electrical conductivity may increase to exhibit conductive characteristics, which may be problematic.

According to one example, the ratio of the metal nanoparticles on the surface of the porous metal-zinc oxide nanosheet may be 1.0 wt % or more.

The ratio of the metal nanoparticles on the surface of the porous metal-zinc oxide nanosheet may be desirably 1.5 wt % or more, and more desirably 2.0 wt % or more.

According to one example, the shape of the metal nanoparticles may include at least one selected from the group consisting of a spherical shape, a hemispherical shape, a prismatic shape, a needle shape, and a plate shape, desirably a hemispherical shape.

According to one example, the method of measuring the diameter of metal nanoparticles may be performed by HR-TEM measurement.

According to one example, the porous metal-zinc oxide nanosheet includes oxygen defects on the surface, and the molar ratio of the oxygen defects may be 5% to 30%.

According to one example, the metal nanoparticles may not only greatly lower energy required to generate oxygen defects, but also stabilize empty spaces. That is, because the oxygen defects act as electron donors, more oxygen defects may be distributed in the surrounding area of the metal nanoparticles.

According to one example, the interfacial interaction between the metal nanoparticles and the porous metal-zinc oxide nanosheet may significantly affect oxygen defect formation due to charge exchange.

According to one example, the molar ratio of the oxygen defects may be 5% to 10%; 5% to 15%; 5% to 20%; 5% to 25%; 5% to 30%; 7% to 15%; 7% to 20%; 7% to 25%; 7% to 30%; 10% to 15%; 10% to 20%; 10% to 30%; 15% to 20%; 15% to 25%; 15% to 30%; and 20% to 30%.

According to one example, when the molar ratio of oxygen defects is less than 5%, the sensitivity of the porous metal-zinc oxide nanosheet may be lowered and thus there may be a problem in detection performance, and when the molar ratio is more than 30%, as the stoichiometric ratio of positive and negative ions in the porous metal-zinc oxide nanosheet is destroyed, repulsive force between the ions is generated to change the lattice structure, thereby causing a decrease in gas sensitivity.

According to one example, the porous metal-zinc oxide nanosheet may further include an electron depletion layer.

According to one example, in the case of a MOS-based gas sensor including the porous metal-zinc oxide nanosheet, the gas sensing response may occur in two steps of 1) adsorption, dissociation, and ionization of oxygen molecules on the surface of the porous metal-zinc oxide nanosheet, and 2) oxidation reaction of the ionized oxygen with the target molecule. In this process, the electron depletion layer may be formed on the surface of the porous metal-zinc oxide nanosheet due to electron consumption for ionizing oxygen atoms. Here, the depth of the electron depletion layer may be modulated in the presence of immobilized metal nanoparticles, and according to structural characterization, oxygen defects may be much high due to the high charge density of the metal nanoparticles. Most of the oxygen defects are located around the metal nanoparticles, so that oxygen molecules may be ionized at these sites. Since more electrons move to oxygen in the porous metal-zinc oxide nanosheet, when there are immobilized metal nanoparticles, a thicker electron depletion layer may be formed, which may mean that the density and size of the metal nanoparticles determine how many electrons are consumed.

For example, small gold nanoparticles densely immobilized on the porous metal-zinc oxide nanosheet may induce oxygen ionization much more easily than large gold nanoparticles sparsely immobilized due to the enormous amount of surrounding empty sites around gold clusters.

According to one example, a thickness ratio of the porous metal-zinc oxide nanosheet and the electron depletion layer may be 1:1 to 8:1.

According to one example, the thickness ratio of the porous metal-zinc oxide nanosheet and the electron depletion layer may be 1:1 to 2:1; 1:1 to 3:1; 1:1 to 4:1; 1:1 to 5:1; 1:1 to 6:1; 2:1 to 5:1; 2:1 to 6:1; 2:1 to 7:1; 3:1 to 6:1; 3:1 to 7:1; and 3:1 to 8:1.

According to one example, when the thickness ratio of the porous metal-zinc oxide nanosheet and the electron depletion layer may is less than 1:1, as the thickness of the electron depletion layer increases, the range of resistance change increases, and sensitivity to gas increases too much, so that there may be a problem in selectivity to gas, and when the thickness ratio is more than 8:1, the thickness of the electron depletion layer decreases and the range of resistance change decreases, so that there may be a problem in reduced sensitivity to gas.

According to one example, the metal nanoparticles may be formed in an area with a depth corresponding to 0% to 10% from the surface of a distance from the surface to the center of the porous metal-zinc oxide nanosheet.

According to one example, the metal nanoparticles may be formed in an area with a depth corresponding to 0% to 1%; 0% to 2%; 0% to 3%; 0% to 4%; 0% to 5%; 0% to 6%; 0% to 7%; 0% to 8%; 0% to 9%; 0% to 10%; 1% to 3%; 1% to 5%; 1% to 10%; 2% to 6%; 2% to 8%; and 2% to 10% from the surface of the distance from the surface to the center of the porous metal-zinc oxide nanosheet.

Accordingly, the metal nanoparticles may be formed only on the surface of the porous metal-zinc oxide nanosheet.

Since the length varies depending on the size of the metal nanoparticles, the length may be limited to the depth from the surface.

According to one example, the metal nanoparticles on the surface of the porous metal-zinc oxide nanosheet may be disposed at intervals of 0 nm to 10 nm.

According to one example, the metal nanoparticles on the surface of the porous metal-zinc oxide nanosheet may be disposed at intervals of 0 nm to 10 nm; 0 nm to 8 nm; 0 nm to 6 nm; 0 nm to 4 nm; 0 nm to 2 nm; 2 nm to 10 nm; 2 nm to 8 nm; 2 nm to 6 nm; 2 nm to 4 nm; 4 nm to 10 nm; 4 nm to 8 nm; 4 nm to 6 nm; 6 nm to 10 nm; or 6 nm to 8 nm.

According to one example, the interval between the metal nanoparticles on the surface of the porous metal-zinc oxide nanosheet may be an interval between the sides of the nanoparticles rather than an interval between the centers of the nanoparticles.

For example, when the metal nanoparticles are disposed at 0 nm intervals on the surface of the porous metal-zinc oxide nanosheet, the metal oxide particles may be disposed tightly without empty spaces.

According to one example, the metal nanoparticles may include at least one selected from the group consisting of Pt, Pd, Rh, Ru, Ni, Co, Cr, Ir, Au, Ag, Pb, Fe, Cu, Al, Ti, Ce, and Cd.

According to one example, the immobilized metal nanoparticles may affect a lattice structure of zinc oxide by inducing several defect sites and oxygen defects. A thicker electron depletion layer is formed due to a large number of oxygen defects, which may contribute to a very large resistance change observed upon exposure to a target molecule.

According to one example, it may be expected that microwave-supported anchoring of the metal nanoparticles on the surface of the porous metal-zinc oxide nanosheet may provide a great aspect for the development of heteroatomic materials for various electrochemical applications in sensing, energy, and catalysis.

According to one example, the pores of the porous metal-zinc oxide nanosheet may be contained in 5 vol % to 30 vol % of the total volume of the porous metal-zinc oxide nanosheet.

According to one example, the pores of the porous metal-zinc oxide nanosheet may be contained in 5 vol % to 10 vol %; 5 vol % to 15 vol %; 5 vol % to 20 vol %; 5 vol % to 25 vol %; 5 vol % to 30 vol %; 10 vol % to 15 vol %; 10 vol % to 20 vol %; 10 vol % to 25 vol %; 10 vol % to 30 vol %; 15 vol % to 30 vol %; 20 vol % to 30 vol %; or 25 vol % to 30 vol % of the total volume of the porous metal-zinc oxide nanosheet.

According to one example, when the pores of the porous metal-zinc oxide nanosheet are contained in less than 5 vol % of the total volume of the porous metal-zinc oxide nanosheet, the inflow and outflow of gas through the pores is too small, diffusion is slowed and thus the responsivity of the gas may decrease. When the pores of the porous metal-zinc oxide nanosheet are contained in more than 30 vol %, gas adsorption in pores is relatively free, so that the sensitivity of the porous metal-zinc oxide nanosheet increases, but recovery time may increase.

According to one example, the pore size of the porous metal-zinc oxide nanosheet may be 0.1 nm to 500 nm.

According to one example, the pore size of the porous metal-zinc oxide nanosheet may be 0.1 nm to 500 nm; 0.1 nm to 400 nm; 0.1 nm to 300 nm; 0.1 nm to 200 nm; 0.1 nm to 100 nm; 0.1 nm to 50 nm; 10 nm to 500 nm; 10 nm to 400 nm; 10 nm to 300 nm; 10 nm to 200 nm; 100 nm to 500 nm; 100 nm to 400 nm; 100 nm to 300 nm; 100 nm to 200 nm; or 200 nm to 500 nm.

A method for manufacturing a porous metal-zinc oxide nanosheet of the present disclosure includes preparing a zinc oxide nanosheet; forming a porous zinc oxide nanosheet by heat-treating the zinc oxide nanosheet; forming a mixture by mixing the porous zinc oxide nanosheet and a metal precursor; and forming metal nanoparticles on the surface of the porous zinc oxide nanosheet by irradiating the mixture with microwaves.

According to one example, the zinc oxide nanosheet may be synthesized using a hydrothermal synthesis method.

According to one example, the molar concentration of the metal precursor may be 0.1 M to 0.5 M. When the molar concentration of the metal precursor exceeds 0.5 M, there may be a problem of agglomerating metal oxide particles. In the mixture prepared with the molar concentration of the metal precursor of 0.1 M to 0.5 M, metal nanoparticles may be formed on the surface of the porous zinc oxide nanosheet when the mixture is irradiated with microwaves.

According to one example, in the forming of the metal nanoparticles on the surface of the porous zinc oxide nanosheet by irradiating the mixture with microwaves, the microwave irradiation may enable uniform heating, uniform nucleation of the metal precursor, and rapid crystal growth.

According to one example, after the forming of the metal nanoparticles on the surface of the porous zinc oxide nanosheet by irradiating the mixture with microwaves, the method may further include completely reducing and immobilizing the metal nanoparticles on the surface of the porous zinc oxide nanosheet by further annealing in an H₂ atmosphere.

According to one example, in the irradiating of the mixture with microwaves, the microwaves may be irradiated at 750 W to 1000 W output for 10 sec to 60 sec.

According to one example, in the irradiating of the mixture with microwaves, the microwaves may be irradiated at 750 W to 800 W output; 750 W to 850 W output; 750 W to 900 W output; 750 W to 950 W output; 750 W to 1000 W output; 800 W to 850 W output; 800 W to 900 W output; 800 W to 950 W output; 800 W to 1000 W output; 850 W to 1000 W output; 900 W to 1000 W output; or 950 W to 1000 W output.

According to one example, in the irradiating of the mixture with microwaves, when the microwaves are irradiated at less than 750 W output, the nucleation density of the metal precursor is lowered and the growth rate of the metal nanoparticles is accelerated, and thus the size of the metal nanoparticles may be increased, and when irradiated at more than 1000 W output, there may be a problem in that the nucleation density of the metal precursor increases and the size of the metal nanoparticles decreases.

According to one example, in the irradiating of the mixture with microwaves, the microwaves may be irradiated for 10 sec to 60 sec; 10 sec to 50 sec; 10 sec to 40 sec; 10 sec to 30 sec; 10 sec to 20 sec; 20 sec to 60 sec; 20 sec to 50 sec; 20 sec to 40 sec; 30 sec to 60 sec; 30 sec to 50 sec; or 40 sec to 60 sec.

According to one example, in the irradiating of the mixture with microwaves, when the microwaves are irradiated for a time in the range, the metal nanoparticles may be densely immobilized on the surface of the porous zinc oxide nanosheet, and suitable conditions to ionize oxygen molecules and form a thick electron depletion layer may be provided.

The gas sensor of the present disclosure includes a chip; and the porous metal-zinc oxide nanosheet formed on the chip.

For example, when the metal nanoparticles of the porous metal-zinc oxide nanosheet are gold, the immobilized gold nanoparticles may use a high affinity between gold and thiyl radicals, and thus the gas sensor may have sulfur selective detection characteristics.

According to one example, the gas sensor may sense at least one selected from the group consisting of a blister agent (HD), 2-CEES, H₂S, CH₄, CO₂, CO, SO₂, NO₂, NH₃, Benzene, Toluene, Xylene, DMMP and 2-CEEC.

For example, when the gas sensor includes the gold-zinc oxide nanosheets and senses 2-CEES, effective adsorption of a 2-CEES product decomposed from the gold nanoparticles may promote a Mars-van Krevelen reaction mechanism for complete oxidation.

According to one example, the gas sensor including the gold-zinc oxide nanosheet may have excellent sensing performance for sulfur compounds.

According to one example, the gas sensor senses gas at a limit concentration of 0.1 ppm at 300° C. to 500° C., and may include a recovery time of 5 sec to 60 sec.

According to one example, the gas sensor may sense the gas at the limit concentration of 0.1 ppm at 300° C. to 500° C.; 320° C. to 500° C.; 330° C. to 500° C.; 350° C. to 500° C.; 380° C. to 500° C.; 400° C. to 500° C.; 420° C. to 500° C.; 440° C. to 500° C.; 460° C. to 500° C.; 480° C. to 500° C.; 300° C. to 350° C.; 300° C. to 400° C.; 300° C. to 450° C.; 350° C. to 400° C.; 350° C. to 450° C.; or 400° C. to 450° C.

According to one example, when the gas sensor is less than 300° C., some of the active sites of the porous zinc oxide nanosheet may be covered with the gold nanoparticles, and these reduced active sites may cause a low response in a low temperature range (<300° C.).

According to one example, when the gas sensor is more than 500° C., the response may be greatly reduced, which may be probably estimated due to the low thermal stability of the gold nanoparticles, sublimation of the porous zinc oxide nanosheet structure and excessive ionization of oxygen atoms, which affect the stoichiometric measurement of chemical oxidation.

According to one example, the gas sensor may include a recovery time of 5 sec to 10 sec; 5 sec to 15 sec; 5 sec to 20 sec; 5 sec to 30 sec; 5 sec to 40 sec; 5 sec to 50 sec; 5 sec to 60 sec; 10 sec to 60 sec; 20 sec to 60 sec; 30 sec to 60 sec; 40 sec to 60 sec; or 50 sec to 60 sec.

According to one example, when the gas sensor has the recovery time in the range, it is possible to obtain highly reliable measurement results as a gas sensor in repeatability evaluation based on repeated measurement of adsorption/desorption, and improve the long-term stability and reproducibility of the gas sensor.

According to one example, the gas sensor may have responsivity of 50 or more at 20% humidity, and responsivity of 30 or more at 80% humidity.

According to one example, when the humidity is 20% or more, the gas sensor may cause poisoning of sensing materials due to atmospheric moisture due to competitive adsorption of water molecules on the surface of the porous metal-zinc oxide nanosheet. The improving of the sensitivity of the gas sensor may be an important factor in overcoming a water pollution effect.

According to one example, when the humidity is 80% or more, the sensing ability may be greatly reduced due to moisture, and when the gas sensor includes the gold-zinc oxide nanosheet, the gas sensor may still have a high response to 2-CEES vapor due to the dramatic improvement in sensitivity by the catalytic effect of the immobilized gold nanoparticles.

Hereinafter, the present disclosure will be described in more detail with reference to Examples and Comparative Examples.

However, the following Examples are just illustrative of the present disclosure, and the contents of the present disclosure are not limited to the following Examples.

It may be reported to apply a microwave-irradiated synthesis of a porous zinc oxide nanosheet immobilized with gold nanoparticles and the sensitive detection of 2-CEES vapor, an analogue of a sulfur blister agent. Upon microwave irradiation for less than 1 minute by annealing, highly crystalline gold nanoparticles with a narrow particle size distribution (2.32±0.40 nm) are densely formed on the surface of the porous zinc oxide nanosheet, which may increase the oxygen defect density of zinc oxide.

At an optimal operating temperature (450° C.), the responsivity of the gas sensor containing the porous gold-zinc oxide nanosheet was measured as 787 for 10 ppm 2-CEES, which was up to 14 times higher than that observed in the gas sensor based on the porous zinc oxide nanosheet. In addition, the porous gold-zinc oxide nanosheet may sense 2-CEES gas even at high humidity (up to 80%) thanks to high sensitivity.

Highly reproducible sensing performance was verified by repeated sensing tests (20 times for 12 hours). According to gas screening data, the porous gold-zinc oxide nanosheet may exhibit excellent selectivity to sulfide compounds due to a high Au—S affinity.

In summary, it is possible to successfully demonstrate a simple and easy approach to form a gold-zinc oxide heterostructure by microwave irradiation and improve gas sensing performance by inducing catalytic activity.

### Example

Zn(NO)₃·6H₂O was dissolved in a mixture of ethanol and water (1:2 v/v), and added with CO(NH₂)₂ (1:1 stoichiometry) with vigorous stirring to obtain a homogeneous mixture. After heating (180° C. for 24 hours), drying (80° C. for 6 hours), and annealing (550° C. for 5 hours under Ar atmosphere), a porous zinc oxide nanosheet was obtained.

To prepare the porous gold-zinc oxide nanosheet through microwave synthesis, 1 at. % of an Au precursor (HAUCl·4H₂O) and 0.8 g of a zinc oxide nanosheet were mixed in 40 mL of DI water-ethanol.

The mixture was placed in an alumina crucible and then treated with microwave irradiation at the output of 1000 W for 1 minute. A commercial microwave oven (Samsung, Model MS32K3513AK) with a frequency of 2.45 GHz was used. The produced pink precipitate was washed several times by centrifugation with DI water and anhydrous ethanol.

Finally, the porous gold-zinc oxide nanosheet was obtained after drying at 80° C. for 6 hours and annealing at 300° C. for 3 hours under a H₂ atmosphere.

### Comparative Example

Zn(NO)₃·6H₂O was dissolved in a mixture of ethanol and water (1:2 v/v), and added with CO(NH₂)₂ (1:1 stoichiometry) with vigorous stirring to obtain a homogeneous mixture. After heating (180° C. for 24 hours), drying (80° C. for 6 hours), and annealing (550° C. for 5 hours under Ar atmosphere), a porous zinc oxide nanosheet was obtained.

### Experiment

The surface and internal structure of a sample were analyzed using scanning electron microscopy (SEM; JEOL-7800F, JEOL Ltd.) and transmission electron microscopy (TEM; JEM-F200, JEOL Ltd.). The microstructure and elemental composition were analyzed using high-angle annular dark-field scanning transmission electron microscopy (HAADF-STEM) and energy-dispersive X-ray spectroscopy (EDS) mapping.

The surface area was calculated using an N₂ adsorption experiment and Brunauer-Emmett-Teller analysis (BET, Autosorb-iQ 2ST/MP, Quantachrome).

The phase and crystal structure were characterized using Cu-Kα radiation (λ=1.5418 Å) by performing X-ray diffraction (XRD; Smart Lab, Rigaku). The chemical binding state was investigated using X-ray photoelectron spectroscopy (XPS; K-alpha, Thermo Fisher Scientific Co.). All XPS profiles were calibrated using a C 1s peak observed at 284.8 eV (C—C bond).

Photoluminescence (PL) and Raman spectroscopy were performed to study lattice defects in the sample using excitation at 325 and 532 nm, respectively (LabRam Aramis and Horriba Jovin Yvon). The electronic structures of the porous zinc oxide nanosheet and the porous gold-zinc oxide nanosheet were measured using ultraviolet photoelectron spectroscopy (UPS) and UV-Vis-NIR spectroscopy (V-650, JASCO Co.).

A commercial MEMS (1 mm², RNS lab) consisting of a Joule heating circuit and drain/source electrodes was used as a chemically resistant gas sensing device. The synthesized porous zinc oxide nanosheet and porous gold-zinc oxide nanosheet were each dispersed in ethanol at a concentration of 1 mg/mL through sonication. 1 μL of the produced dispersion was drop cast several times on a heated MEMS chip placed on a 100° C. hot plate. Each electrode was connected to a ceramic package via a gold wire using a wire bonder (HB-05, TPT Wire Bonder GmbH & Co. KG).

A heating circuit of the MEMS was connected to a DC power supply device (SDP30-5D, SM techno), and then heated to a specific temperature (200 to 500° C.) by controlling a Joule heating voltage (1.0 to 2.5 V). The resistance of the sensing material was monitored using a digital source meter/source measurement device (SMU, Keithley 2450, Keithley Instruments) under a constant voltage of 1 V.

A standard gas cylinder containing 20 ppm 2-CEES balanced with N₂ was used as a target gas.

The gas flow rate was automatically controlled using a customized gas exposure system consisting of a Mass Flow Controller (MFC) and LabView software. Various concentrations of 2-CEES gas were prepared by diluting 20 ppm 2-CEES gas with synthetic air at a desired ratio.

To measure a sensor response in humid conditions, humid air was prepared by bubbling through distilled water and mixing dry synthetic air in a specific ratio.

### Results and Discussion

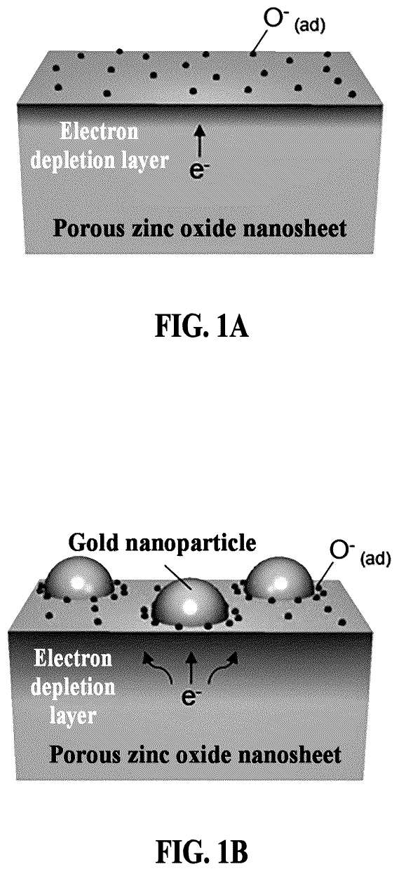

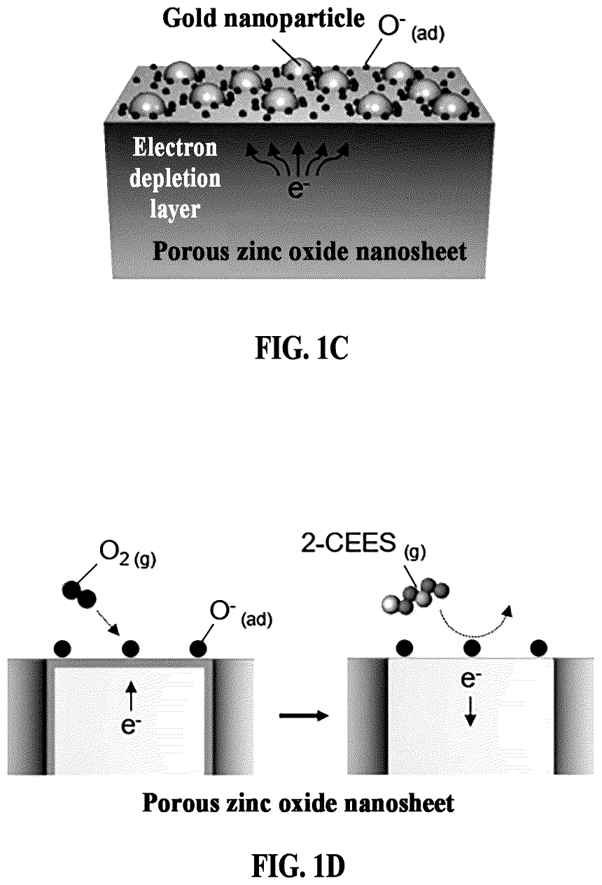

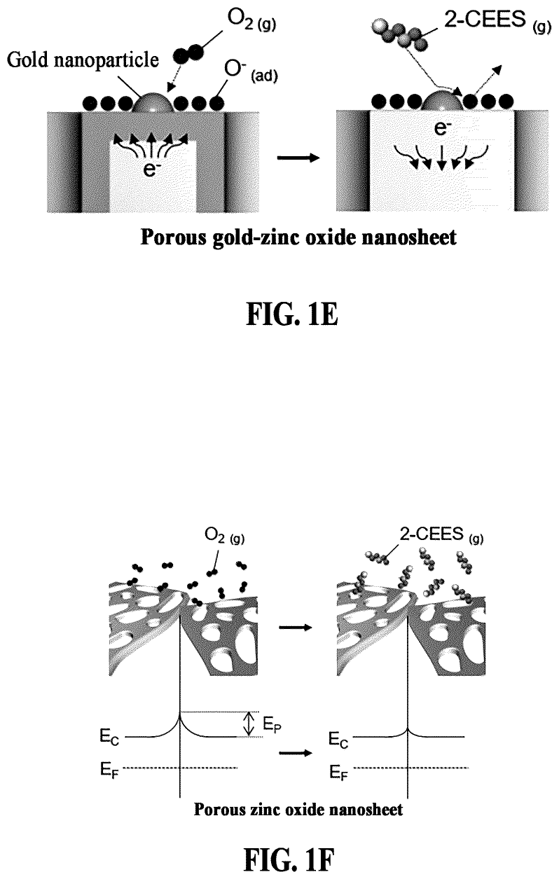

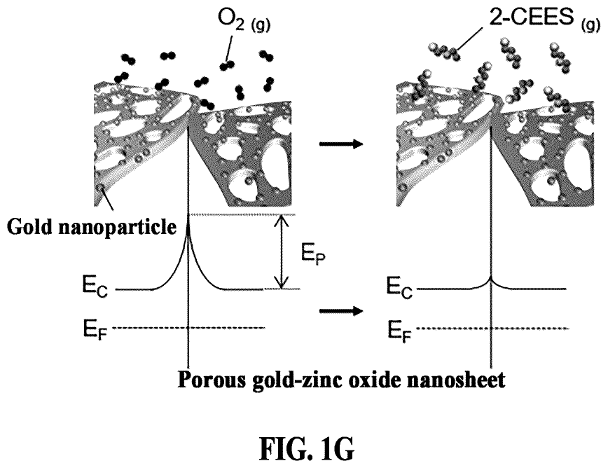

FIG. 1 is a schematic diagram illustrating a gas sensing mechanism according to Example and Comparative Example of the present disclosure; FIG. 1A a state without gold nanoparticles, FIG. 1B a state in which large gold nanoparticles are formed at a rare density, FIG. 1C a state in which nano-sized gold nanoparticles are densely immobilized, FIG. 1D a schematic diagram illustrating a gas sensing mechanism on the surface of a porous zinc oxide nanosheet, FIG. 1E a schematic diagram illustrating a gas sensing mechanism on the surface of a porous gold-zinc oxide nanosheet, FIG. 1F changes in potential barrier of a porous zinc oxide nanosheet, and FIG. 1G changes in potential barrier of a porous gold-zinc oxide nanosheet.

In the case of an MOS-based gas sensor, the gas sensing reaction may occur in two steps of 1) adsorption, dissociation, and ionization of oxygen molecules on the oxide surface and 2) oxidation reaction of ionized oxygen with a target molecule.

In the first step, when the porous zinc oxide nanosheet was exposed to air, gaseous oxygen (O_{2(g)}) may diffuse to the metal oxide surface and be physically adsorbed (O_{2(ads)}). As the temperature increases, these adsorbed oxygen molecules may receive electrons from the oxide and be chemically adsorbed (O₂⁻_{(ads)}). At a high temperature oxide surface (>300° C.), oxygen molecules may be dissociated and then dissociated and ionized to O⁻_{(ads)} and O₂⁻_{(ads)} by receiving more electrons from oxygen defects.

The oxygen ionization process on the metal oxide surface may be described using the following Equations (2) to (5).

$${O}_{2 ( g )} \to {O}_{2 ( ads )} \qquad ( 2 )$$

$${O}_{2 ( ads )} + {e}^{-} \to {{O}_{2}^{-}}_{( ads )} \qquad ( 3 )$$

$${{O}_{2}^{-}}_{( ads )} + {e}^{-} \to 2 {{O}^{-}}_{( ads )} \qquad ( 4 )$$

$${{O}^{-}}_{( ads )} + {e}^{-} \to {{O}^{2 -}}_{( ads )} \qquad ( 5 )$$

In this process, an electron depletion layer may be formed on the oxide surface due to the consumption of electrons to ionize oxygen atoms. Here, the depth of the electron depletion layer may be modulated in the presence of immobilized gold nanoparticles, and FIG. 1 may illustrate FIG. 1A a state without gold nanoparticles, FIG. 1B a state in which large gold nanoparticles are formed at a rare density, and FIG. 1C a state in which nano-sized gold nanoparticles are densely immobilized.

According to structural characterization, Example has much higher oxygen defects than Comparative Examples due to the high charge density of a noble metal. Most of the oxygen defects are located around the gold nanoparticles, so that oxygen molecules may be ionized at these sites.

Since more electrons are transferred from the oxide to oxygen, when there are immobilized gold nanoparticles, a thicker electron depletion layer may be formed, which may mean determining how many electrons are consumed depending on the density and size of the gold nanoparticles.

For example, densely immobilized small gold nanoparticles may induce oxygen ionization much more easily than sparsely immobilized large gold nanoparticles due to the enormous amount of surrounding empty sites around the gold clusters.

In Example, only gold nanoparticles of 2.3 nm in size are densely immobilized on the oxide surface, which may provide suitable conditions for ionizing oxygen molecules and forming a thick electron depletion layer.

FIG. 1 illustrates FIG. 1D a schematic diagram illustrating a gas sensing mechanism on the surface of Comparative Example, and FIG. 1E a schematic diagram illustrating a gas sensing mechanism on the surface of Example. When 2-CEES gas reacts with ionized oxygen, the electrons consumed for oxygen ionization are regenerated and returned to a conduction band of the metal oxide, so that the thickness of the electron depletion layer may rapidly decrease.

For the oxidation reaction, 2-CEES molecules may be dissociated into two radical compounds according to Equation (6) below. Thiyl radicals (R—S·) interact strongly with the gold surface to be easily adsorbed on the immobilized gold nanoparticles. The oxidation of the adsorbed 2-CEES occurs through the Mars-van Krevelen mechanism, which may be a reaction between adsorbed species of the gold nanoparticles and ionized oxygen in the peripheral regions of the gold nanoparticles.

According to this process, the reaction kinetics may be expressed as Equations (7) and (8) below. After oxidation, products CO₂, SO₂ and H₂O may be desorbed from the sensing material.

$${ClCH}_{2} {CH}_{2} {SCH}_{2} {CH}_{3} \to {ClCH}_{2} \cdot + {CH}_{3} {CH}_{2} S \cdot \qquad ( 6 )$$

$$2 {ClCH}_{2} {CH}_{2} \cdot + {80}^{-} \to 2 {CO}_{2} + {Cl}_{2} + 4 {H}_{2} O + 8 {e}^{-} \qquad ( 7 )$$

$$2 {CH}_{3} {CH}_{2} S \cdot + {130}^{-} \to 2 {SO}_{2} + 5 {H}_{2} O + 2 {CO}_{2} + 13 {e}^{-} \qquad ( 8 )$$

FIG. 1 illustrates (f) changes in potential barrier of Comparative Example, and (g) changes in potential barrier of Example, and changes in the electron depletion layer may affect the potential barrier at a junction between the nanosheet particles.

When a voltage is applied to an electrode, a mobile charge carrier in one nanosheet may move to another adjacent charge carrier along the electric field. However, if a junction potential barrier is too large so as not to be overcome, the overall sensor resistance may increase as charge carriers may not move.

Comparative Example has a thin electron depletion layer due to a low amount of oxygen defects to have a relatively small potential barrier and a low reference resistance. Even if Comparative Example reacts with 2-CEES molecules, the sensor resistance may not change significantly because only a small amount of reaction sites is present.

On the other hand, Example has a thick electron depletion layer due to many oxygen defects, which may generate a large potential barrier and generate a high reference resistance at the particle junction. When a gas sensor including Example is exposed to 2-CEES gas, the 2-CEES molecules may efficiently react with a huge amount of ambient oxygen ions around the gold nanoparticles through an Au—S interaction and a Mars-van-Krevelen reaction mechanism. It may be shown that the resistance value decreases dramatically by significantly reducing the thicknesses of the electron depletion layer and the gold nanoparticles.

In summary, the immobilization of gold nanoparticles on the oxide may significantly improve a sensor response by increasing the thickness of the electron depletion layer and the junction potential barrier and promoting oxygen adsorption, dissociation, and ionization.

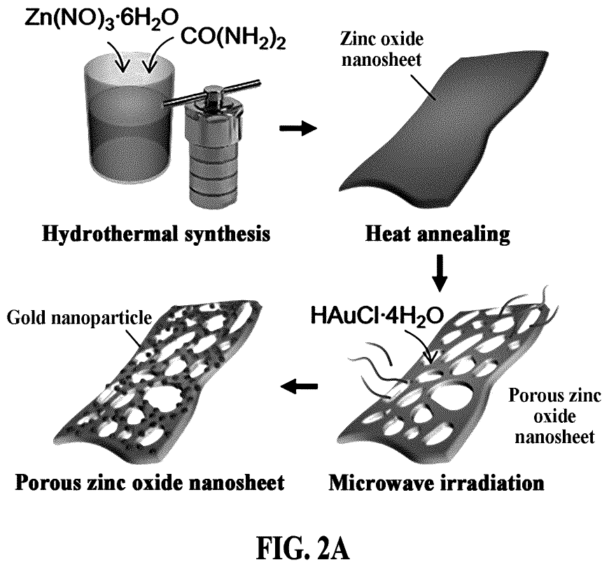

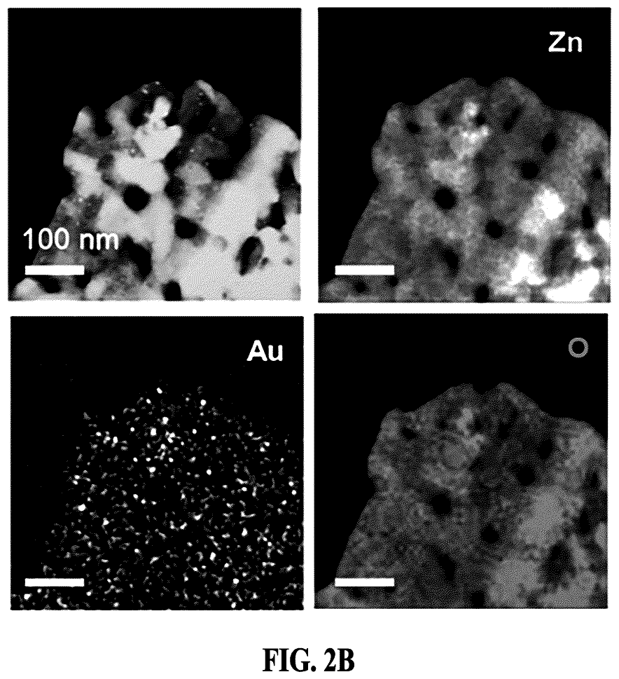

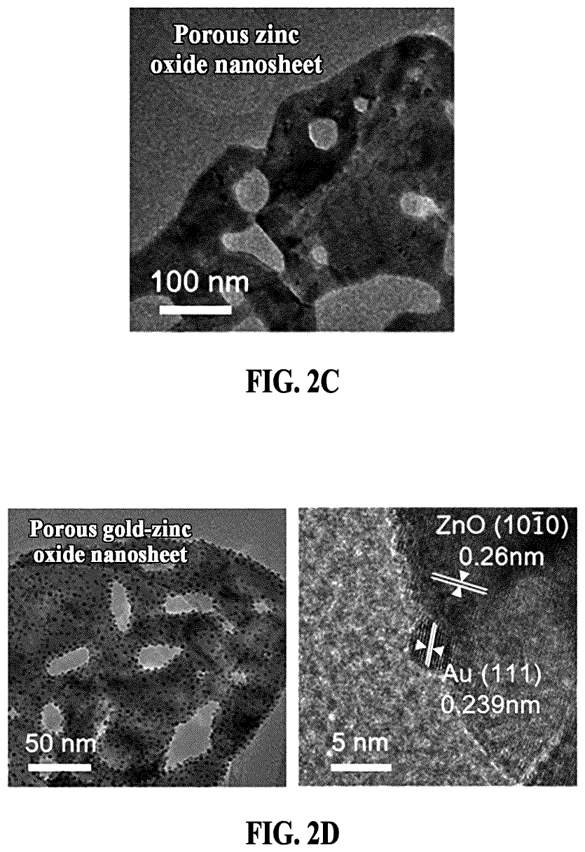

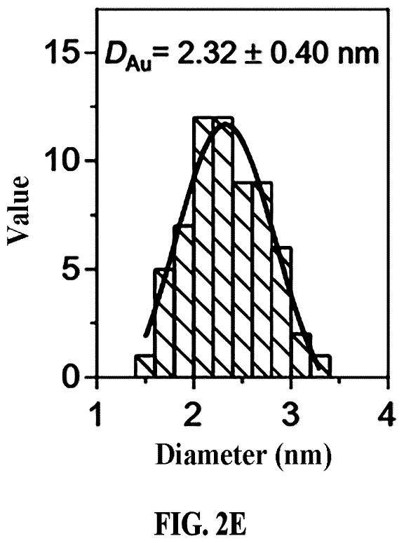

FIG. 2 illustrates FIG. 2A a schematic diagram of a manufacturing process of a porous gold-zinc oxide nanosheet containing gold nanoparticles formed on the surface, FIG. 2B a surface element analysis result of a porous gold-zinc oxide nanosheet using energy-dispersive X-ray spectroscopy, FIG. 2C a high-resolution transmission electron microscopy image of a porous zinc oxide nanosheet, FIG. 2D a high-resolution transmission electron microscopy image of a porous gold-zinc oxide nanosheet, and FIG. 2E a size distribution of gold nanoparticles formed on the surface of a porous gold-zinc oxide nanosheet, according to Example and Comparative Example of the present disclosure.

The manufacturing process of Example was illustrated in FIG. 2A, and the zinc oxide nanosheet was successfully synthesized by hydrothermal synthesis in an autoclave chamber. To maximize the effective surface area for gas adsorption, a zinc oxide nanosheet was annealed at 550° C. under an Ar atmosphere to generate a porous morphology. As a catalytic site for gas adsorption and electrochemical reaction, gold nanoparticles were introduced by irradiating microwaves to a homogeneous mixture of a gold precursor and a porous zinc oxide nanosheet. Then, the product was further annealed in a H₂ atmosphere to achieve complete reduction and immobilization of gold nanoparticles on the surface of the porous zinc oxide nanosheet.

FIG. 2B may observe decorated gold nanoparticles in a TEM image using surface element analysis as a result of surface element analysis of Example using energy dispersive X-ray spectroscopy (EDS), which may show that gold nanoparticles are densely distributed on the surface of the porous zinc oxide nanosheet.

When analyzing how microwave irradiation affected the formation of gold nanoparticles compared to a product without microwave treatment, the results may show that gold nanoparticles with a scale of less than 1 nm have been formed due to nucleation from the gold precursor by rapid and homogeneous energy transfer on the surface of the porous zinc oxide nanosheet immediately after microwave treatment.

These gold nanoparticles and the remaining unreacted gold precursor may be aggregated and firmly immobilized on the surface of the porous zinc oxide nanosheet when annealed in H₂ atmosphere for several hours.

The shape, crystallinity, and particle size distribution of gold nanoparticles may be observed as a high-resolution transmission electron microscopy image of a porous zinc oxide nanosheet in FIG. 2C, and a high-resolution transmission electron microscopy image of a porous gold-zinc oxide nanosheet in FIG. 2D. FIGS. 2C and 2D may show that the hemispherical shape of the gold nanoparticles is clearly observed as a highly crystalline lattice structure. Fringe spacings of 0.25 and 0.239 nm correspond to the lattice planes of zinc oxide (101̅0̅) and Au (111), respectively, which may indicate that gold nanoparticles are immobilized on the surface of the porous zinc oxide nanosheet.

FIG. 2E illustrates a size distribution of gold nanoparticles formed on the surface of Example, and the size distribution of gold nanoparticles is 2.32±0.40 nm. Well-structured gold nanoparticles may be stably immobilized on the surface of the porous zinc oxide nanosheet, thanks to the most energetically stable interface formed between Au (111) and zinc oxide (101̅0̅).

Gold nanoparticles produced by hydrothermal synthesis in an autoclave chamber have relatively large sizes (up to 50 nm) with a low density, whereas gold nanoparticles using microwaves have advantages in terms of a simple experimental procedure, a narrow particle size distribution, and high-density decoration.

The study of heterogeneous structures in Example may be well established based on various approaches.

As shown in Table 1 below, the synthesis method is useful in terms of simple experimental procedures, fast reaction rate, high density, and uniform particle size control.

TABLE 1

| Morphology of | Synthesis | | | | |
|---|---|---|---|---|---|
| zinc oxide | method | Reagent | Particle size | | Application |
| Ladder-like | Annealing(H₂) | HAuCl₄•4H₂O | Au single atom | | Gas sensor |
| structures | | | | | |
| Nano-rod | Chemical | HAuCl₄•3H₂O, | To 15 nm | | UV |
| | method | Na₃C₆H₅O₇•2H₂O | | | photodetector |
| Cylindrical | Hydrothermal | NaBH4, HAuCl₄ | To 4 nm | | Photocatalyst |
| structure | | | | | |
| Nano-wires | Deposition- | HAuCl₄Na₂CO₃ | 6.3 ± 3 | nm | Catalytic |
| | precipitation | | | | oxidation |
| | method | | | | |
| Porous nano- | Microwave & | HAuCl₄•4H₂O | 2.32 ± 0.40 | nm | Gas sensor |
| sheets | annealing(H2) | | | | |

Since the interfacial binding between the noble metal and the oxide support acts as an ‘electron exchange window’ and induces structural defects in the oxide lattice, changes in chemical state of the porous zinc oxide nanosheet in the presence of immobilized gold nanoparticles were further examined.

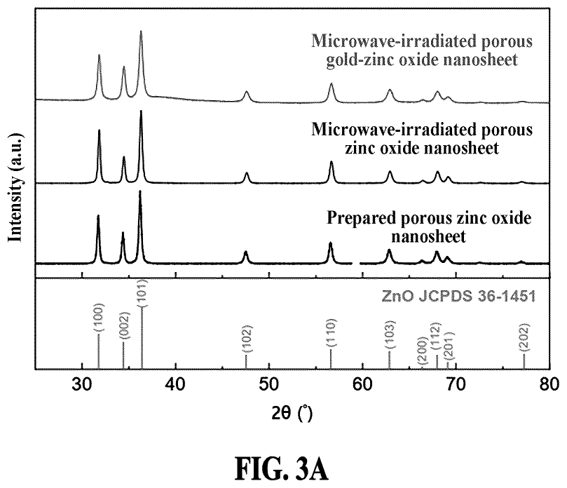

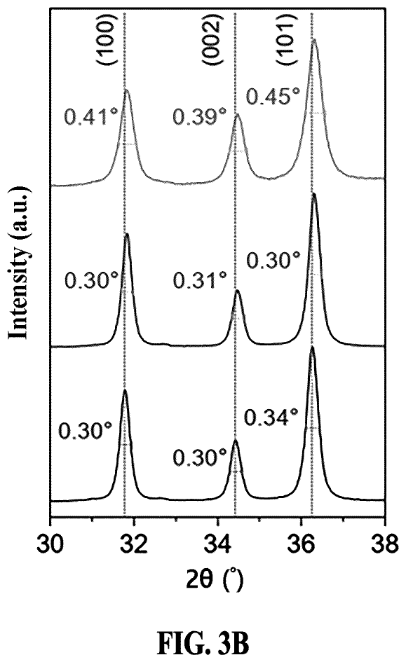

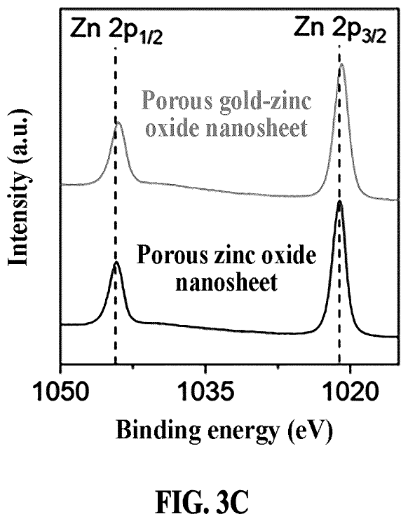

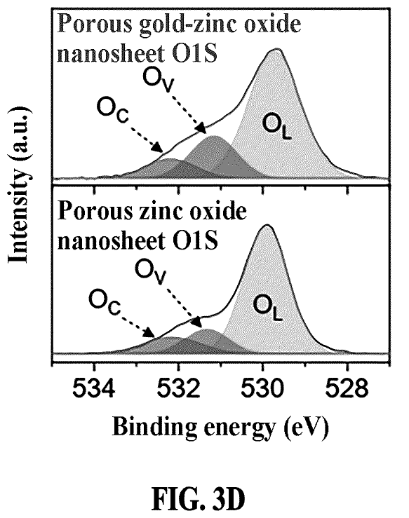

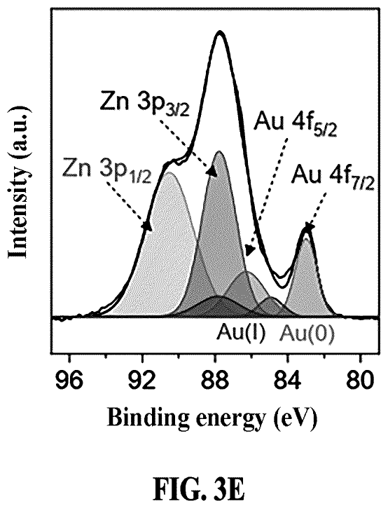

FIG. 3 illustrates FIG. 3A crystallographic analysis results of a porous gold-zinc oxide nanosheet and a porous zinc oxide nanosheet using X-ray diffraction, FIG. 3B a result of chemical bond state analysis of a porous zinc oxide nanosheet and a porous gold-zinc oxide nanosheet obtained from movement of zinc oxide lattice position after microwave irradiation, peak expansion after immobilization of gold nanoparticles, and high-resolution X-ray photoelectron spectroscopy, FIG. 3C zinc binding energy before and after gold nanoparticle immobilization, FIG. 3D an oxygen binding state of a porous zinc oxide nanosheet and a porous gold-zinc oxide nanosheet, and FIG. 3E a binding state of gold nanoparticles immobilized on the surface of the porous gold-zinc oxide nanosheet, according to Example and Comparative Example of the present disclosure.

To clarify the effect of microwave irradiation, XRD spectra of porous zinc oxide nanosheets treated with microwaves without a gold precursor were collected.

FIG. 3A illustrates XRD patterns obtained for Example and Comparative Example, which may clearly show a single phase of hexagonal Wurtzite zinc oxide phase (JCPDS No. 36-1451).

FIG. 3B illustrates a result of chemical bond state analysis of a porous zinc oxide nanosheet and a porous gold-zinc oxide nanosheet obtained from movement of zinc oxide lattice position after microwave irradiation, peak expansion after immobilization of gold nanoparticles, and high-resolution X-ray photoelectron spectroscopy. An impurity phase was not detected according to a pattern, but the (100), (002), and (101) diffraction peaks of the microwave-treated Example and the porous zinc oxide nanosheet were shifted to a higher angle than those observed in Comparative Example. The shift of the XRD peaks may be attributed to the stress in the unit cell in the crystal structure due to the presence of oxygen defect-rich ions on the interface of the porous zinc oxide nanosheet of the oriented attachment. In other words, since the electromagnetic field of the microwaves induced dipole polarization of the zinc oxide lattice, the energy may be converted to defect sites that generated heat.

In addition, it was shown that the full width at half maximum of the peak was widened after immobilization of the gold nanoparticles, which may mean that the gold nanoparticles form an Au—O—Zn heteroatom junction interface to weaken the crystallinity of the zinc oxide lattice structure.

The changes in the zinc oxide lattice structure were further studied by analyzing the chemical binding states for Zn, O, and Au using XPS, and FIG. 3C illustrates Zn binding energy before and after gold nanoparticle immobilization, in which Zn 2p_{1/2} and 2p_{3/2} states, representing Zn—O binding states, were clearly observed at 1044.22 and 1021.12 eV, respectively.

In Example, the binding energy value of the Zn 2p state also slightly decreased to about 0.31 eV compared to Comparative Example, which may be because the electron density around Zn atoms has been improved.

FIG. 3D illustrates oxygen binding states of Example and Comparative Example, and the effect of immobilized gold nanoparticles on the Zn—O binding state was also determined by deconvolution O is spectra, Zn—O lattice oxygen (O_{L}, to 530 eV), empty oxygen (O_{V}, to 531 eV), and chemisorbed oxygen (O_{C}, to 532 eV).

Changes in oxygen state in Example and Comparative Example are summarized in Table 2 below. Surprisingly, the O_{V} ratio was higher after immobilizing the gold nanoparticles.

Previous density functional theory studies have reported that when noble metals were in close contact with a metal oxide surface, the noble metal clusters tended to capture more electron density, which not only significantly lowered the energy required for the metal to generate oxygen defects, but also stabilized empty spaces. In other words, since the oxygen defects act as electron donors, more oxygen defects may be distributed in the surrounding area of the noble metal cluster.

In the present disclosure, hemispherical gold nanoparticles are tightly immobilized on the surface of the porous zinc oxide nanosheet to provide ideal conditions for the formation of oxygen defects.

FIG. 3E illustrates a binding state of gold nanoparticles immobilized on the surface of Example, and binding energies of Zn 3p and Au 4f observed in Example were analyzed and deconvoluted to several components corresponding to Zn 3p_{1/2} (90.51 eV), Zn 3p_{3/2} (87.77 eV), Au 4f_{5/2} (86.99 eV) and Au 4f_{7/2} (83.02 eV). The Au 4f signal was also deconvoluted into ionic (Au⁺) and metallic (Au0) phases, which may suggest the presence of an Au—O—Zn interface.

In particular, the binding energies of Au 4f_{5/2} and Au 4f_{7/2} were downshifted by 0.72 and 0.98 eV, respectively, compared to bulk Au (Au 4f_{5/2}=87.71 and Au 4f_{7/2}=84.00 eV).

The shift of the Au signal also confirmed the presence of a large electron density around gold atoms, derived from oxygen defects formed in the zinc oxide lattice.

In summary, the interfacial interaction between gold nanoparticles and zinc oxide may have a significant effect on oxygen defect formation due to charge exchange.

TABLE 2

| Sample | O_{L} (eV, %) | O_{V} (eV, %) | O_{C} (eV, %) |
|---|---|---|---|
| Bare porous zinc | 529.92 | 531.32 | 532.17 |
| oxide NSs | (73.32%) | (14.43%) | (12.25%) |
| Au-zinc oxide NSs | 529.71 | 531.16 | 532.18 |
| | (69.01%) | (21.02%) | (9.97%) |

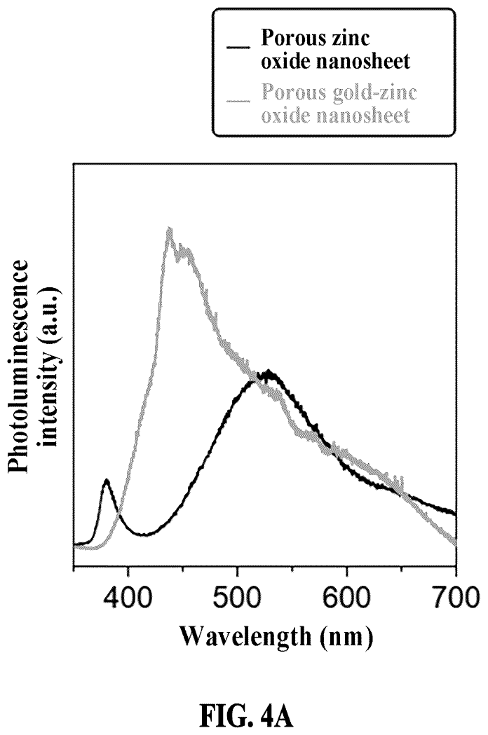

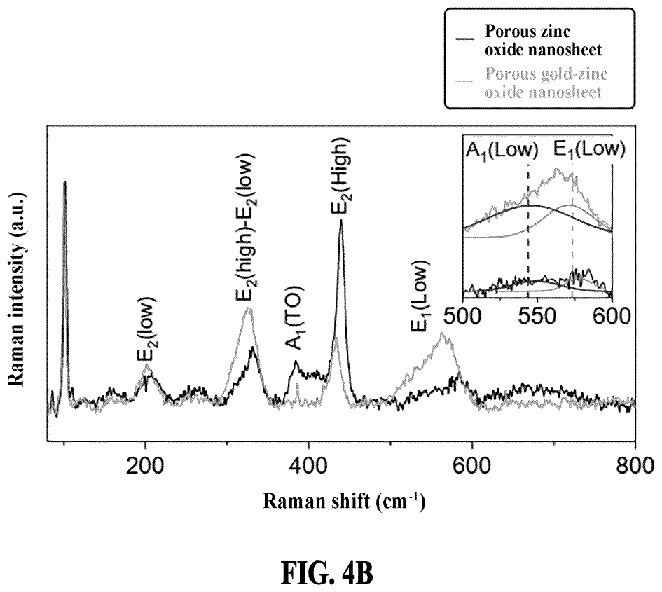

FIG. 4 illustrates FIG. 4A a result of measuring photoluminescence (PL) of a porous zinc oxide nanosheet and a porous gold-zinc oxide nanosheet and FIG. 4B a result of Raman shift observation after immobilizing gold nanoparticles, according to Example and Comparative Example of the present disclosure. Defect formation in Example may be confirmed using Raman spectroscopy.

FIG. 4A illustrates photoluminescence spectra obtained in Example and Comparative Example, and PL measurements were closely related to the degree of defects corresponding to O_{V} in MOS and thus compared.

Both the signal intensity and the accumulated signal area were increased in Example, which may indicate that the degree of defect has increased.

In addition, the Raman spectra of Example and Comparative Example were measured at an excitation laser wavelength of 532 nm.

In Comparative Example, a Wurtzite phase vibration mode of zinc oxide was mainly observed at 438 cm⁻¹ [E₂(high)].

The overall Raman shift of Example in the range of 100 to 800 cm⁻¹ showed a slight red shift due to weakened Zn—O bonds, which consistent with a decrease in lattice parameters observed in XRD analysis. A shift of the peak position to a lower wave number was observed, which may indicate the occurrence of structural changes.

As compared to Comparative Example, the broad and weak peak observed at up to 438 cm⁻¹ [E₂(high)] may also be allocated to a low crystal quality of the P63mc symmetry of the Wurtzite structure due to higher defect and stress densities of Example.

In addition, an E₁(LO) phonon mode observed at up to 581 cm⁻¹ may represent defects in the zinc oxide lattice, such as gap defects (Znᵢ) or O_{V}.

FIG. 4B illustrates a result of Raman shift observation after immobilizing gold nanoparticles, and this peak was deconvoluted using Lorentz fitting with the A₁(LO) peak at up to 557 cm⁻¹, which was associated with the O_{V} defects.

As a result, gold nanoparticle immobilization may affect the zinc oxide lattice structure and generate many defect sites.

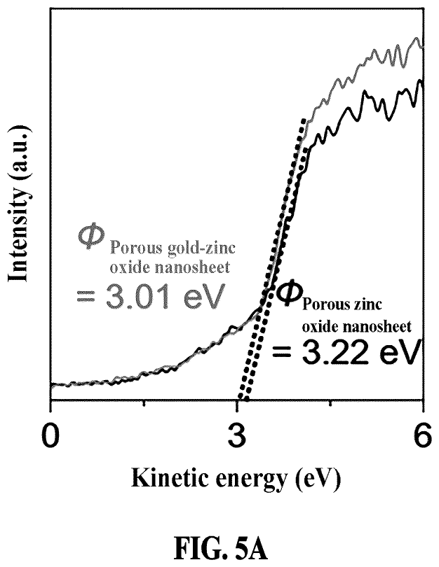

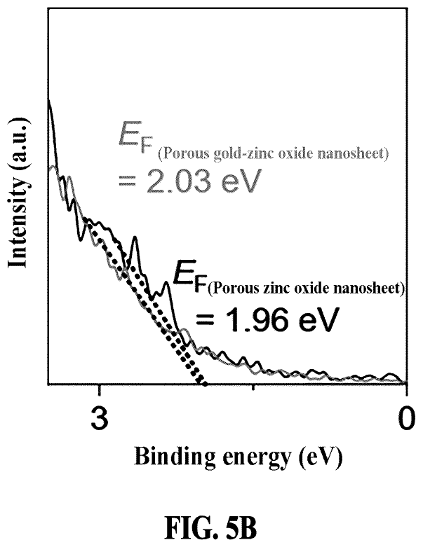

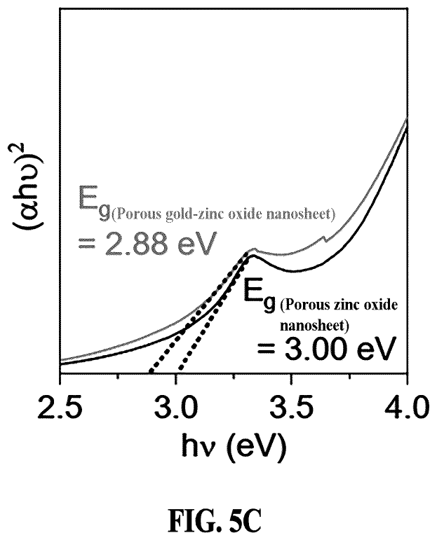

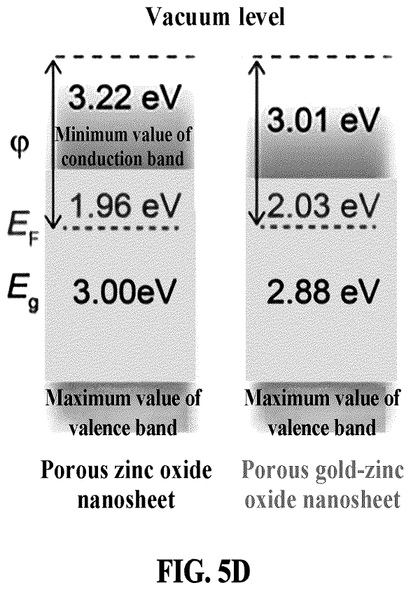

FIG. 5 illustrates results of analyzing bandgap structures of a porous zinc oxide nanosheet and a porous gold-zinc oxide nanosheet according to Example and Comparative Example of the present disclosure; FIG. 5A kinetic energy of emitted electrons, FIG. 5B binding energy of electrons measured by ultraviolet photoelectron spectroscopy, FIG. 5C a band gap energy measurement result calculated from Tauc-plot obtained by UV-vis-NIR spectroscopy, and FIG. 5D expected band structures of a porous zinc oxide nanosheet and a porous gold-zinc oxide nanosheet.

FIG. 5A illustrates kinetic energy of emitted electrons, and FIG. 5B illustrates binding energy of electrons measured by ultraviolet photoelectron spectroscopy. Here, changes in band structure after immobilizing the gold nanoparticles were analyzed, and work functions (Φ) and Fermi levels (E_{F}) of Example and Comparative Example were measured using UPS.

FIG. 5C illustrates a band gap energy measurement result calculated from Tauc-plot obtained by UV-vis-NIR spectroscopy, and the band gap energy (E_{g}) was calculated by measuring the Tauc-plot, which was an edge of the absorption profile, using UV-vis NIR spectroscopy.

The E_{F} of Example (2.03 eV) was slightly higher than the E_{F} of Comparative Example (1.96 eV), which may indicate an electron doping effect due to oxygen defects and strong n-type transport characteristics.

The work function (Φ) values were measured as 3.22 and 3.01 eV for Comparative Example and Example, respectively, which indicates that electrons may be emitted more easily in Example than in Comparative Example.

In addition, E_{g} of Example (2.88 eV) was also decreased compared to Comparative Example (3.00 eV), which may be because a high level of oxygen defects has generated an impurity energy level near the valence band energy level.

FIG. 5D illustrates energy band diagrams of a porous zinc oxide nanosheet and a porous gold-zinc oxide nanosheet based on the measurement.

The modulated band structure of Example may clearly show that immobilized gold nanoparticles generate high density surface electrons at oxygen defects, resulting in a strong n-type component.

Since the degree of oxygen defects was one of the main factors of determining sensing performance, gas sensing characteristics of Examples were examined. Many studies have reported that materials were modified through doping, surface chemistry, and size control to increase the number of oxygen defects and dramatically improve sensitivity.

In the present disclosure, an increase in the number of oxygen defects is confirmed by forming a noble metal/oxide heterogeneous structure, so that it may be expected that Example will exhibit better sensing characteristics than Comparative Example.

A MEMS-based chemically resistant gas sensor was fabricated and a gas exposure experiment was performed in a customized gas chamber system.

Briefly, Comparative Example and Example were dispersed in ethanol and drop cast onto an MEMS heated until drain/source electrodes were sufficiently connected by a sensing material.

The operating temperature of the gas sensor was controlled through Joule heating when a voltage was applied to a thermal induction circuit. The generated heat was quickly transferred to the sensing material of a measuring electrode within a few seconds. Since a reference resistance and a signal amplitude were easily affected by the temperature, the sensor response was normalized using Equation (1) below.

$$Response = \frac{{R}_{a} - {R}_{g}}{{R}_{g}} = \frac{Δ R}{{R}_{g}} \qquad ( 1 )$$

In which, Rₐ and R_{g} represent average resistance levels when exposed to a reference air atmosphere and 2-CEES, respectively. Changes in electrical conductivity upon gas exposure were calculated.

Before performing the gas exposure experiment, the electronic properties of the deposited sensing material were characterized.

It was found that Comparative Example and Example showed different reference resistance tendencies depending on a Joule heating temperature.

Generally, when MOS is heated, electrons in a valence band are excited in a conduction band, which may increase electrical conductivity.

This phenomenon was observed when the temperature was increased from 200° C. to 500° C. at 50° C. intervals in the sensor including Comparative Example.

On the other hand, the resistance level of the sensor including Example gradually decreased in a low temperature range (<250° C.), but increased above 300° C.

This difference may be interpreted as a difference in potential energy barrier of a conduction path resulting from the electron depletion layer.

It is known that chemically adsorbed oxygen molecules are dissociated and ionized to O⁻ at 100 to 300° C., and begin to be ionized to O²⁻ at a higher temperature by obtaining electrons from oxygen defects. In this process, since the electrons of the oxide are transferred to oxygen atoms, an electron depletion layer may be formed on the oxide surface.

In other words, the more oxygen defects exist on the metal oxide surface, the more electrons may be extracted from the conduction band of the oxide, thereby generating a thicker electron depletion layer and a higher potential barrier.

It may be seen that the gold nanoparticles induced many oxygen defects in the lattice of the porous zinc oxide nanosheet due to a high electron charge density.

It may be inferred that Example has a much thicker electron depletion layer than Comparative Example.

In particular, the resistance of Examples starts to increase at 300 to 400° C., which may mean that the ionization of oxygen molecules is promoted by a catalytic effect of the immobilized gold nanoparticles in this temperature range.

As a result, mobile charge carriers in the nanosheet may not freely move to another nanosheet due to such an energy barrier.

The electron depletion layer was also formed in Comparative Example, but the depth of the potential barrier seemed to be negligible due to the small number of oxygen defects.

It was analyzed how gold nanoparticles contributed to sensing performance by measuring the sensor response to 2-CEES, which was an analogue of a blister agent (HD) CWA.

Previous studies have reported excellent sensitivity and selective detection of sulfur compounds using a SERS effect of gold nanoparticles.

Heterogeneous catalysis studies also reported that oxidation reactions through the Mars-van Krevelen mechanism occurred between molecules adsorbed on noble metal species and ambient oxygen around the immobilized metal clusters.

Therefore, it may be assumed that Example also has sensitivity to sulfide compounds due to high catalytic activity.

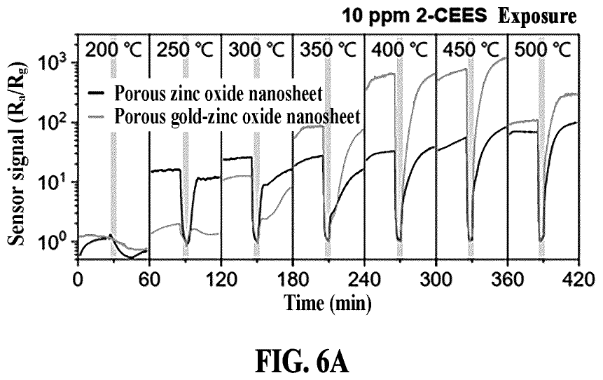

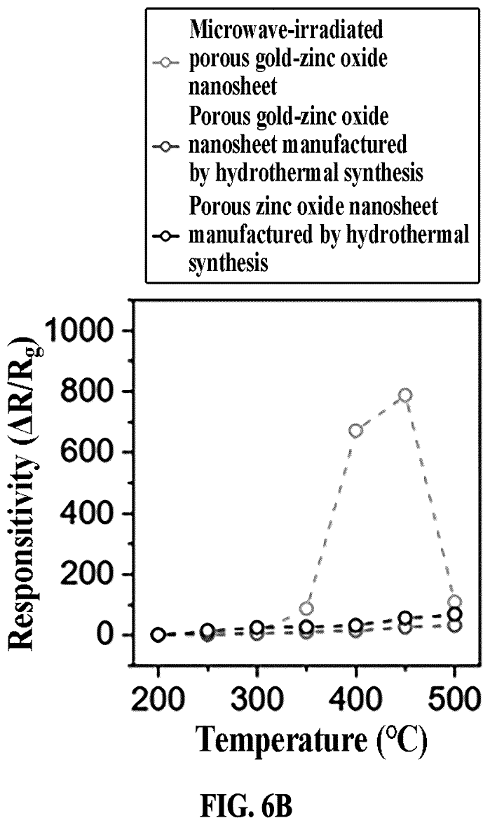

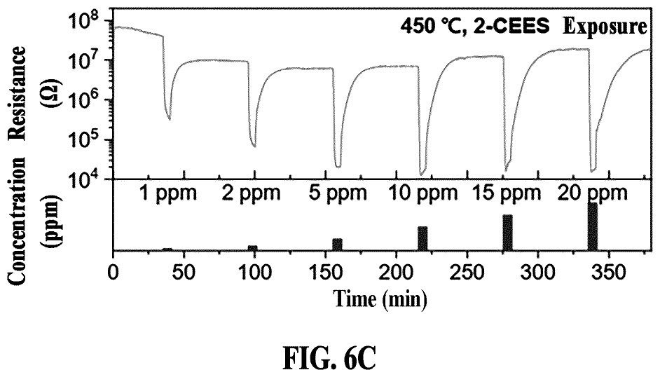

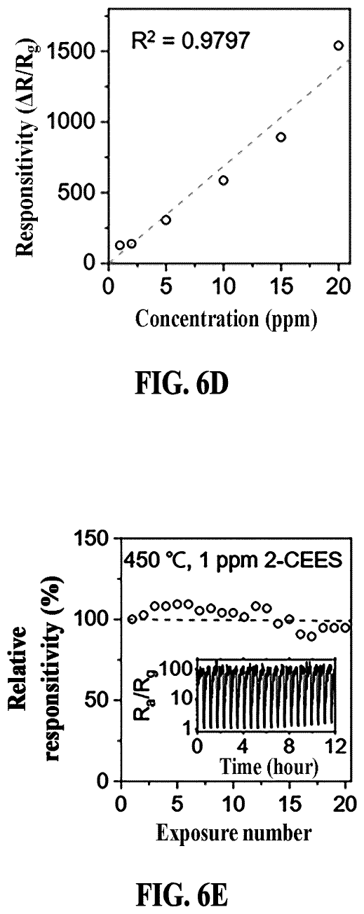

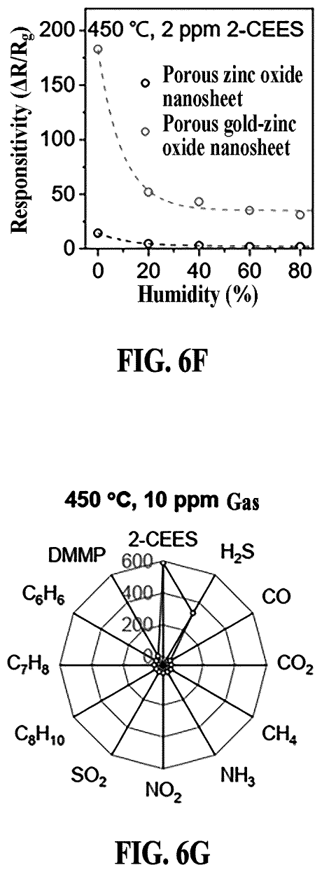

FIG. 6 illustrates FIG. 6A a temperature-dependent resistance change curve upon exposure to 10 ppm 2-CEES as a result of evaluating gas sensing performance of a porous zinc oxide nanosheet and a porous gold-zinc oxide nanosheet, FIG. 6B a result of summarizing gas responsivity of a porous zinc oxide nanosheet and a porous gold-zinc oxide nanosheet produced by a hydrothermal synthesis method and a porous gold-zinc oxide nanosheet irradiated with microwaves, FIG. 6C a resistance change curve of a gas sensor including a porous gold-zinc oxide nanosheet exposed to various concentrations of 2-CEES gas by gas concentration-dependent gas sensing performance of the gas sensor including the porous gold-zinc oxide nanosheet, FIG. 6D gas responsivity according to a gas concentration obtained through FIG. 6C, FIG. 6E a result of evaluating sensing performance reproducibility by repeating gas exposure 20 times for 12 hours, FIG. 6F a result of measuring gas sensing performance of gas sensors including a porous gold-zinc oxide nanosheet and a porous zinc oxide nanosheet under various humidity conditions, and FIG. 6G sensing selectivity of gas sensors including porous gold-zinc oxide nanosheets for various gaseous compounds, according to Example and Comparative Example of the present disclosure.

A gas sensing experiment was performed using sulfur compounds, and a temperature dependent gas sensing test was performed to determine the optimal operating temperature of Example for 2-CEES sensing. FIG. 6A illustrates a temperature-dependent resistance change curve upon exposure to 10 ppm 2-CEES as a result of evaluating gas sensing performance of Comparative Example and Example.

The manufactured gas sensor was exposed to 10 ppm 2-CEES using a customized gas exposure system after stabilizing the reference resistance. In the case of an n-type zinc oxide-based gas sensor, detection of 2-CEES was shown in a rapid decrease in resistance. FIG. 6B may illustrate a result of summarizing gas responsivity of a porous zinc oxide nanosheet and a porous gold-zinc oxide nanosheet produced by a hydrothermal synthesis method and a porous gold-zinc oxide nanosheet irradiated with microwaves. This result may show that both Comparative Example and Example may not detect 2-CEES gas at 200° C., but show different reaction tendencies at a high temperature.

The response of the sensor including Comparative Example gradually increased from 250° C. to 500° C., but the response of the gas sensor including Example may be shown as a pyramid profile with a maximum response at 450° C.

The response of the sensor including Comparative Example was similar to or slightly higher than that of the gas sensor including Example in a low temperature range (<300° C.).

However, the response of the gas sensor including Example begins to be improved dramatically at 300° C. or more, which may exhibit about 14 times higher value (≈787) than that observed for the sensor (≈56) including Comparative Example at 450° C.

As a control group of Example synthesized by irradiating microwaves, a porous gold-zinc oxide nanosheet manufactured by hydrothermal synthesis was tested, but detection enhancement could be not observed due to non-immobilized gold nanoparticles with low density and large size to the metal oxide.

Through measurements, a correlation between the reference resistance level and the sensor response may be observed. As described above, the immobilized gold nanoparticles may promote the formation of oxygen defects to generate a thicker electron depletion layer and reduce electrical conductivity.

Under these conditions, 2-CEES is more likely to react with ionized oxygen in oxygen defects, and promotes electron exchange between 2-CEES and the metal oxide, thereby causing dramatic changes in resistance. This mechanism seemed to be applicable at a temperature of 300° C. or more when the resistance of Example began to increase.

On the other hand, the gas detection mechanism of Example may be similar to that of Comparative Example in a low operating temperature range (<300° C.) where the catalytic effect was minimal.

As a BET result, it may be confirmed that the volume of pores is decreased after gold nanoparticles are immobilized on the surface of the porous zinc oxide nanosheet. In other words, some of the active sites of the porous zinc oxide nanosheet may be covered with gold nanoparticles. These reduced active sites may cause a low response of Example in the low temperature range (<300° C.).

Meanwhile, when the temperature is 450° C. or more, the response decreases greatly, which may be probably estimated due to the low thermal stability of gold nanoparticles, sublimation of the structure of the porous zinc oxide nanosheet, and excessive ionization of oxygen atoms, which affect the stoichiometric measurement of chemical oxidation.

After determining the optimal temperature, additional quantitative analysis was performed according to a gas concentration.

For the experiment, various concentrations of 2-CEES gas (1, 2, 5, 10, 15, and 20 ppm) were prepared by diluting 20 ppm of 2-CEES standard gas with synthetic air using MFC.

The sensor was exposed to target gas sequentially from a low concentration to a high concentration to measure sensor resistance. FIG. 6C illustrates a resistance change curve of a gas sensor including a porous gold-zinc oxide nanosheet exposed to various concentrations of 2-CEES gas by gas concentration-dependent gas sensing performance of the gas sensor including the porous gold-zinc oxide nanosheet.

The amplitude of the sensor signal gradually increases at a high concentration of 2-CEES gas.

FIG. 6D illustrates gas responsitivity according to a gas concentration obtained through FIG. 6C, and the sensor response obtained for each concentration was plotted and it was found that the least square regression analysis of the linear fitting matches the data very well (R²=0.9797). Based on the fitting curve, the sensitivity of the gas sensor including Example was calculated to be up to 68.81 ppm⁻¹.

FIG. 6E illustrates a result of evaluating sensing performance reproducibility by repeating gas exposure 20 times for 12 hours, and the reproducibility of detection ability was estimated by exposing the sensor to 1 ppm 2-CEES gas 20 times for 12 hours. When each response was normalized to an initial response, the linear regression fitting curve shows a slope of 5×10⁻⁴, which may mean that the reproducibility of the sensor is high despite repeated measurements for a long period of time.

Table 3 below summarizes the reported sensing characteristics associated with 2-CEES sensing.

Although previous studies on quantum dot (QD) materials showed higher sensing responses than this study, the QD materials were less stable after exposure to high temperature conditions.

The present disclosure may demonstrate excellent sensing performance in terms of maintaining high sensitivity for long-term measurement.

TABLE 3

| | Operating | Gas | | | | |
|---|---|---|---|---|---|---|
| Sensing | temperature | concentration | Response | Response | Recovery | |
| material | (° C.) | (ppm) | (ΔR/R₈) | time (s) | time (s) | Reference |
| Au—ZnO NS | 450 | 10 | 787 | 27 | 822 | This study |
| WO₃/WS₂ | 240 | 5.7 | 0.81 | 20 | 55 | [60] |
| hetero structures | | | | | | |
| Al-doped | 450 | 20 | 5393 | 3 | 406 | [48] |
| ZnO QDs | | | | | | |
| ZnO QDs | 450 | 20 | 4614 | 3 | 207 | [48] |
| Al-doped | 500 | 20 | 954 | 2 | 127 | [61] |
| ZnO NP | | | | | | |
| ZnO NP | 500 | 20 | 344 | 6 | 165 | [61] |
| Sm₂O₃ doped | 200 | 10 | 540 | 40 | 20 | [62] |
| SnO₂ NP | | | | | | |
| SnO₂ NP | 250 | 10 | 180 | 50 | 20 | [62] |
| Ru—CdSnO₃ | 350 | 4 | 62 | 5 | 185 | [63] |
| thin film | | | | | | |
| Pt—CdSnO₃ | 250 | 4 | 59 | 30 | 300 | [64] |
| thin film | | | | | | |
| CdSnO₃ | 350 | 4 | 12 | 2 | 75 | [64] |
| thin film | | | | | | |

In order to confirm the effectiveness of Example for sensing of a CWA gas sensor, the humidity-dependent function needs to be verified because CWA is mainly used in outdoor environments.

One of difficult problems for the MOS-based gas sensor may be poisoning of the sensing material by atmospheric moisture due to competitive adsorption of water molecules on the metal oxide surface.

Many approaches have been developed to minimize water poisoning, such as integration of hydrophilic packed-bed columns, deposition of a hydrophobic layer, surface functionalization of oxides with a moisture-resistant hydrophobic polymer, and coating of a metal-organic structure.

These methods are effective for moisture poisoning, but coated functional materials typically require high temperatures for oxidation of complex molecules, which may limit the operating conditions of the sensing material. Therefore, the improving of the sensitivity of the sensor may be an important factor in overcoming a water pollution effect.

To evaluate the sensing performance under various humid conditions, the humidity level was controlled by diluting moisture-saturated air with dry air.

FIG. 6F illustrates a result of measuring gas sensing performance of gas sensors including a porous gold-zinc oxide nanosheet and a porous zinc oxide nanosheet under various humidity conditions, and the concentration of 2-CEES gas was fixed to 2 ppm, and sensing performance was measured at 0, 20, 40, 60, and 80% of humidity.

When the humidity was 20%, both the sensors including Example and Comparative Example were significantly poisoned by moisture, and responsivity was reduced by 30% (52 in Example, 4.71 in Comparative Example) compared to dry conditions (183 in Example, 14.25 in Comparative Example).

At high humidity (80%), the response further decreased to 31 in Example and 2.18 in Comparative Example, and Example showed at least 15 times higher response than Comparative Example.

Although the sensing ability was greatly reduced by moisture, Example still showed a high response to 2-CEES vapor due to a dramatic improvement in sensitivity due to the catalytic effect of the immobilized gold nanoparticles.

FIG. 6G illustrates sensing selectivity of gas sensors including porous gold-zinc oxide nanosheets for various gaseous compounds. To identify false positive malfunctions of a sensor for interfering gas, with respect to generally observed carbon oxidation compounds (CO and CO₂), hydrocarbons (CH₄, benzene (C₆H₆)), toluene (C₇H₈) and xylene (C₈H₁₀), nitrogen compounds (NH₃ and NO₂), sulfur compounds (H₂S and SO₂), and DMMP, a nerve agent-like agent, the sensing selectivity of Examples was examined and the gas concentration was fixed at 10 ppm.

The highest response was observed for H₂S (324), followed by DMMP (14.7), CH₄ (6.85), C₇H₈ (6), and NH₃ (1.67).

The gas sensor including Example has excellent selectivity to 2-CEES gas except H₂S, which is excellent considering that the response to 10 ppm 2-CEES is up to 587. This high selectivity to sulfur compounds may be described by a high interaction affinity between the gold surface and sulfur atoms.

In particular, when a sulfhydryl group is deprotonated, thiyl radicals (R—S·) are generated, which may form covalent bonds with the Au surface. Even if no radicals are generated, thiol groups (R—SH) may have coordination-type bonding interactions with the Au surface through lone pair electrons in sulfur.

It is known that a 2-CEES molecule dissociates into two radicals at high temperatures and terminates with a sulfur radical. The sulfur radical may interact actively with the Au (111) surface, so that the molecule may react efficiently with lattice oxygen. In the case of SO₂, sulfur is already completely oxidized, so that oxidation may not occur on the oxide surface.

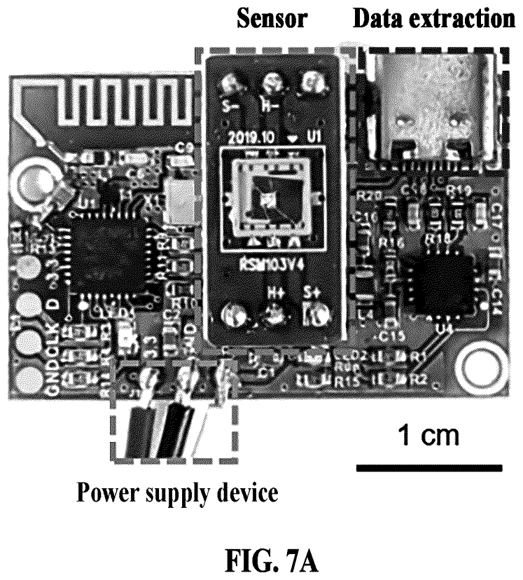

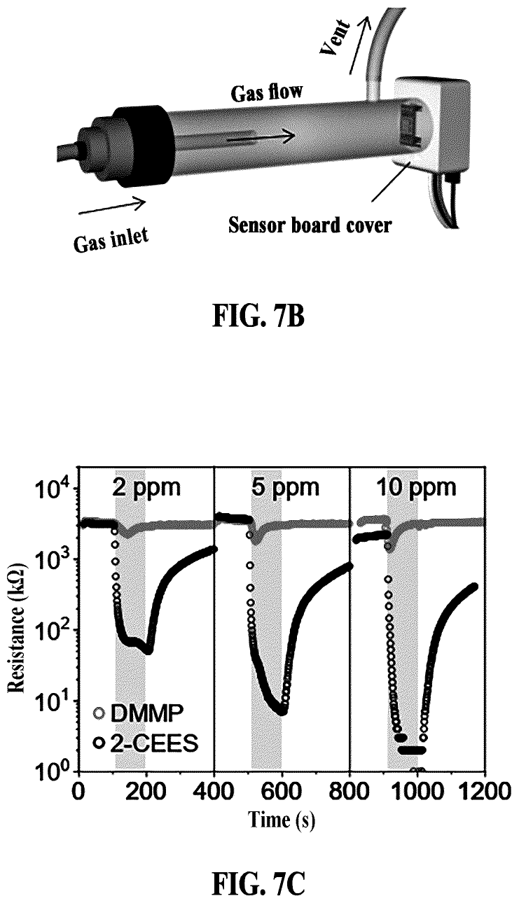

FIG. 7 illustrates FIG. 7A a photograph of a portable sensor module with dimension 34 mm×22 mm and weight 4.82 g, FIG. 7B a brief diagram of a gas sensing experiment, and FIG. 7C a resistance change curve of gas sensors including porous gold-zinc oxide nanosheets in various concentrations of 2-CEES and DMMP, as evaluation of sensing performance of a porous gold-zinc oxide nanosheet mounted on a portable sensor board according to Example and Comparative Example of the present disclosure.

To verify the sensing function in a field environment, a portable sensor board integrated with a developed sensor package was designed, and FIG. 7A shows a photograph of a portable sensor module with dimension 34 mm×22 mm and weight 4.82 g. The dimension of the board was 34 mm×22 mm and the weight was 4.82 g, which had little effect on the mobility of a remotely controlled robotic system such as drones. The operating temperature of the sensor was controlled by an external power supply device (2.0 V) connected to electrodes on a printed circuit board (PCB). To measure a resistance level of the sensor, a constant voltage (3.3 V) was applied to the sensor circuit by another power source. The sensor resistance value was measured by monitoring the divided potential up to a certain resistance connected in series to the sensor circuit. The sensor module was operated by receiving commands through a serial program and communicating with a laptop. The measured data were automatically stored in an internal memory, which could be extracted to a laptop via a wired connection. Before gas exposure, the sensor module was assembled into a customized cover package to prevent mechanical damage from external stresses.

FIG. 7B shows a brief diagram of a gas sensing experiment, and target gas was prepared by the same gas exposure system and flowed through a quartz tube.

FIG. 7C illustrates a resistance change curve of gas sensors including porous gold-zinc oxide nanosheets in various concentrations of 2-CEES and DMMP, and through the system, various concentrations of gas were exposed to the sensor module. According to the extracted data, the gas sensor including Example showed responses of 62, 527, and 1087 at 2, 5, and 10 ppm of 2-CEES gas, respectively, which may be similar to the values measured with high-performance multimeter equipment. In addition, the sensor barely responded to DMMP gas, and the values of DMMP gas were 1.26 at 2 ppm, 1.96 at 5 ppm, and 2.72 at 10 ppm.

In summary, excellent sensitivity and selectivity for 2-CEES were confirmed and the multifunctionality of the gas sensor in a portable module through measurement was confirmed.

### CONCLUSION

The present disclosure may report the microwave synthesis of a heterogeneous structure, such as gold nanoparticles immobilized on the porous zinc oxide nanosheet, and easy application as a CWA gas sensor with high sensitivity and selectivity for 2-CEES gas. Compared with conventional hydrothermal synthesis methods, a simple microwave irradiation approach may provide high-density decoration, narrow particle size distribution, and strong structural immobilization of gold nanoparticles on the zinc oxide surface within 1 minute. The immobilized gold nanoparticles may affect the lattice structure of zinc oxide by inducing several defect sites and oxygen defects. A thicker electron depletion layer is formed due to a large number of oxygen defects, which may contribute to a very large resistance change observed upon exposure to a target molecule. The immobilized gold nanoparticles may also impart sulfur-selective detection characteristics by applying a high affinity between Au (111) and thiyl radicals. In addition, effective adsorption of a decomposed 2-CEES product on the gold nanoparticles may promote a Mars-van Krevelen reaction mechanism for complete oxidation.

In the present disclosure, it may be expected that microwave-supported anchoring of noble metals on the surface of metal oxide may provide an excellent aspect for the development of heteroatomic materials for various electrochemical applications in sensing, energy, and catalysis.

As described above, although the examples have been described by the restricted drawings, various modifications and variations may be applied on the basis of the embodiments by those skilled in the art. For example, even if the described techniques are performed in a different order from the described method, and/or components such as a system, a structure, a device, a circuit, etc. described are coupled or combined in a different form from the described method, or replaced or substituted by other components or equivalents, an appropriate result may be achieved.

Therefore, other implementations, other examples, and equivalents to the appended claims fall within the scope of the claims to be described below.

What is claimed is:

1. A porous metal-zinc oxide nanosheet comprising metal nanoparticles formed on the surface.

2. The porous metal-zinc oxide nanosheet of claim 1, wherein a diameter of the metal nanoparticles is 0.1 nm to 5 nm, and
a ratio of the metal nanoparticles on the surface of the porous metal-zinc oxide nanosheet is 1.0 wt % or more.

3. The porous metal-zinc oxide nanosheet of claim 1, wherein the porous metal-zinc oxide nanosheet comprises oxygen defects on the surface, and a molar ratio of the oxygen defects is 5% to 30%.

4. The porous metal-zinc oxide nanosheet of claim 1, further comprising:
an electron depletion layer.

5. The porous metal-zinc oxide nanosheet of claim 4, wherein a thickness ratio of the porous metal-zinc oxide nanosheet and the electron depletion layer is 1:1 to 8:1.

6. The porous metal-zinc oxide nanosheet of claim 1, wherein the metal nanoparticles are formed in an area with a depth corresponding to 0% to 10% from the surface of a distance from the surface to the center of the porous metal-zinc oxide nanosheet.

7. The porous metal-zinc oxide nanosheet of claim 1, wherein the metal nanoparticles on the surface of the porous metal-zinc oxide nanosheet are disposed at intervals of 0 nm to 10 nm.

8. The porous metal-zinc oxide nanosheet of claim 1, wherein the metal nanoparticles comprise at least one selected from the group consisting of Pt, Pd, Rh, Ru, Ni, Co, Cr, Ir, Au, Ag, Pb, Fe, Cu, Al, Ti, Ce, and Cd.

9. The porous metal-zinc oxide nanosheet of claim 1, wherein pores of the porous metal-zinc oxide nanosheet are comprised in 5 vol % to 30 vol % of the total volume of the porous metal-zinc oxide nanosheet.

10. A method for manufacturing a porous metal-zinc oxide nanosheet comprising:
preparing a zinc oxide nanosheet;
forming a porous zinc oxide nanosheet by heat-treating the zinc oxide nanosheet;
forming a mixture by mixing the porous zinc oxide nanosheet and a metal precursor; and
forming metal nanoparticles on the surface of the porous zinc oxide nanosheet by irradiating the mixture with microwaves.

11. The method for manufacturing the porous metal-zinc oxide nanosheet of claim 10, wherein in the irradiating the mixture with microwaves,
the microwaves are irradiated at 750 W to 1000 W output for 10 sec to 60 sec.

12. A gas sensor comprising:
a chip; and
the porous metal-zinc oxide nanosheet of any one of claims 1 to 9 formed on the chip.

13. The gas sensor of claim 12, wherein the gas sensor senses at least one selected from the group consisting of a blister agent (HD), 2-CEES, H₂S, CH₄, CO₂, CO, SO₂, NO₂, NH₃, Benzene, Toluene, Xylene, DMMP and 2-CEEC.

14. The gas sensor of claim 12, wherein the gas sensor senses gas at a limit concentration of 0.1 ppm at 300° C. to 500° C., and comprises a recovery time of 5 sec to 60 sec.

15. The gas sensor of claim 12, wherein the gas sensor has responsivity of 50 or more at 20% humidity, and responsivity of 30 or more at 80% humidity.
